(19) United States
(12) Patent Application Publication
(10) Pub. No.: US 2019/0329188 A1
(43) Pub. Date: Oct. 31, 2019

(54) MEMBRANE FOR THE ADSORPTION OF BACTERIA

(71) Applicant: GAMBRO LUNDIA AB, Lund (SE)

(72) Inventors: Benjamin MALARD, Villette d'Anthon (FR); Nathalie PHILIPPE, Genas (FR)

(21) Appl. No.: 16/343,542

(22) PCT Filed: Nov. 29, 2017
(86) PCT No.: PCT/EP2017/080860

§ 371 Date: Apr. 19, 2019

(30) Foreign Application Priority Data
Nov. 29, 2016 (EP) 16201096.1

(51) Int. Cl. B01D 71/42; B01D 67/00; B01D 69/02; B01D 69/06; B01D 69/08; B01D 69/10; A61M 1/36

(52) U.S. Cl. CPC B01D 71/42; B01D 67/0093; B01D 69/02; B01D 69/06; B01D 69/08; B01D 69/10; B01D 61/243; B01D 2325/36; B01D 2325/48; B01D 2325/16; B01D 2325/14; B01D 2325/12; A61M 1/3679

(57) ABSTRACT

The present disclosure relates to semipermeable membranes based on acrylonitrile copolymers capable of adsorbing bacteria from fluids, methods of producing such membranes, and devices comprising such membranes.

### TECHNICAL FIELD

The present disclosure relates to semipermeable membranes based on acrylonitrile copolymers capable of adsorbing bacteria from fluids, methods of producing such membranes, and devices comprising such membranes.

### DESCRIPTION OF THE RELATED ART

The presence of microbial pathogens in the bloodstream is a well-known contributor to sepsis. Persistence of both living and dead pathogens due to inadequate antibiotic response (complexity of germ identification, antibiotic-resistant pathogens) is a major clinical issue. It is hypothesized that a blood purification technique that would allow for the removal of living pathogens might represent a beneficial adjunctive therapy to the administration of antibiotics.

The use of semipermeable membranes based on acrylonitrile copolymers for blood treatment is known. For example, membranes made from the acrylonitrile-sodium methallylsulfonate copolymer, called AN69, are commercially available. A review of the state of the art for AN69 membranes can be found in Thomas et al., Contrib Nephrol. 2011; 173:119-29.

European patent application EP 0 925 626 A1 describes a device for the treatment of blood or plasma by extracorporeal circulation, comprising a semi-permeable membrane based on polyacrylonitrile carrying bound negative charges wherein, before or after formation of the membrane, at least one neutral or cationic polymer is incorporated into the membrane, in a suitable quantity so as to regulate the overall ionic capacity and the electrokinetic index of the membrane, in a suitable manner. The polymer may be cationic and selected from polyamines, preferably from polyethyleneimines.

US 2003/0021826 A1 proposes binding, in a stable manner to the surface of semi-permeable membranes essentially comprised of a copolymer of acrylonitrile and at least one anionic and anionizable monomer, an anticoagulation agent which can exert its anticoagulating activity without being leached out into the blood or plasma during treatment by extracorporeal circulation to reduce the quantity of anticoagulation agent used systemically in the patient during an extracorporeal blood treatment session. A semi-permeable composite membrane is disclosed which comprises a semi-permeable support membrane and an anticoagulation agent suitable for the treatment of blood or plasma by extracorporeal circulation, said semi-permeable support membrane being essentially constituted by a polyacrylonitrile carrying anionic or anionizable groups; the surface of the semi-permeable support membrane intended to be brought into contact with the blood or plasma is coated in succession with a cationic polymer carrying cationic groups which can form an ionic bond with anionic or anionizable groups of polyacrylonitrile, the cationic polymer (for example polyethyleneimine, PEI) comprising chains of a size which is sufficient not to traverse the semi-permeable support membrane, and an anticoagulation agent carrying anionic groups which are capable of forming an ionic bond with cationic groups of said cationic polymer (for example heparin).

WO 2007/148147 A1 describes the use, on a membrane preferably based on a copolymer of acrylonitrile and sodium methallylsulfonate, of a solution of a polymer carrying anionic or anionizable groups in the colloidal form and in an acidic medium, in particular by mixing, for example, a solution of polymer carrying anionic or anionizable groups with a solution of organic polyacid in a specific proportion with respect to said polymer, which results in an increase in both the quantity of polymer grafted to the surface of the membrane and the availability of free cationic or cationizable groups at the surface of this membrane coating. The membrane described thus allows a large quantity of compounds carrying anionic or anionizable groups to be bound. A method for preparing the membrane is also described in WO 2007/148147 A1.

Methods for preparing acrylonitrile-based membranes are also disclosed in U.S. Pat. No. 5,626,760 A. Methods for producing a hydrogel copolymer comprising acrylonitrile and methallylsulfonate are disclosed, for example, in DE 689 13 822 T2.

The acrylonitrile-based membranes described above have been successfully used for adsorbing endotoxins contained in biological fluids, e.g., blood or plasma. However, their ability to remove living pathogens, i.e., bacteria, is unsatisfactory.

### SUMMARY

It is an object of the present disclosure to provide a membrane for removing living pathogens, i.e., bacteria, from a liquid. The membrane is based on a copolymer of acrylonitrile and sodium methallylsulfonate and is coated with at least one cationic polymer in colloidal form. It is also an object of the present disclosure to provide a device for the removal of bacteria from a liquid, e.g., a biological fluid such as blood, plasma or another liquid which may be injected into a patient, for instance, water, aqueous solutions of electrolytes or drugs, or liquids for parenteral nutrition. Methods for removing bacteria from a liquid and methods for producing a device for the removal of bacteria from a liquid also are provided.

In a further aspect, the disclosure provides a device for the removal of bacteria from blood or plasma and for the treatment of renal insufficiency (in particular by hemodialysis, ultrafiltration, hemofiltration and/or hemodiafiltration).

In a still further aspect, the disclosure provides an improvement to the semi-permeable membrane and device of WO 2007/148147 A1, ensuring an excellent adsorption capacity for bacteria contained in the fluid intended to be filtered through said membrane.

### BRIEF DESCRIPTION OF THE DRAWINGS

FIG. 1 is a schematic representation of a setup for determining bacteria adsorption on the membrane of a dialyzer.

FIG. 2 illustrates the results of an evaluation of the adsorption of Staphylococcus aureus on different hollow fiber membranes, as further described in the Examples.

FIG. 3 shows removal of Staphylococcus aureus from human plasma by a PEI-grafted AN69 membrane as a function of PEI concentration on the membrane, as further described in the Examples.

### DETAILED DESCRIPTION

Membranes based on acrylonitrile copolymers have long since been known in the art and are commercially available, for example the membranes often collectively referred to as “AN69” membranes. In the context of the present disclosure, the term “AN69 membrane” or “AN69 type membrane” refers to membranes based on a copolymer of sodium methallylsulfonate and acrylonitrile. The AN69 membranes are known for their high water content of up to 73%. In the present disclosure, the values recited for water content of membranes refer to the equilibrium water content of the respective membrane under ATPS conditions, i.e., at ambient temperature and ambient pressure, and in air saturated with water vapor.

One example for a current product comprising a AN69 type membrane is the Evodial dialyzer, which is a hemodialyzer equipped with a heparin-grafted acrylonitrile based membrane such as described in the aforementioned WO 2007/148147 A1 (the so-called HeprAN membrane). The Evodial membrane is characterized also in that the charged surface, originating from anionic sulfonate groups, is neutralized by the polycationic biopolymer polyethylenimine (Thomas et al. (2011), Contrib Nephrol. Basel, Karger, vol 173, 119-129). The surface treatment also allows the almost irreversible fixing of said heparin through very strong ionic binding between the negative charges of heparin and the free positive charges of the cationic polymer. Membranes having the ability to immobilize heparin are highly desirable as it further reduces the need of systemic doses of heparin, and might even allow heparin-free dialysis possible especially for patients with high risk of bleeding (Thomas et al., Contrib Nephrol. 2011; 173:119-29).

The disclosure is based on an optimized coating of the AN69 membrane using a PEI polycationic polymer in a colloidal form, as documented in WO 2007/148147 A1. The membrane, in addition to endotoxin removal, allows for removal of bacteria without triggering undesirable clotting side effects. In contrast to the membrane taught by WO 2007/148147 A1, the membrane of the present disclosure is not coated with heparin. The membrane will preferably be used in combination with citrate anticoagulation, so that no risks related to adsorption of heparin used intravenously by the membrane can occur. An advantage of the membrane of the present disclosure is the large spectrum of its mode of action (removal of fluid, inflammatory mediators, and endotoxins, in addition to bacteria removal).

It is an important feature of the membrane that it provides an adequate balance between the amount of free amine (—NH₂) groups at the surface allowing bacteria adsorption, while preventing undesirable blood activation cascade. A proper equilibrium is particularly important in the absence of heparin, which at the same time minimizes the amount of free (—NH2) groups at the surface and exerts a potent anticoagulation effect via ATIII binding. A membrane having an inadequate excess of amine (—NH₂) surface charge is undesirable and may lead to premature blood coagulation by hydrogen bonds with fibrinogen which further may activate platelets. It has been shown that surfaces having free amine (—NH₂) groups notably adsorb more fibrinogen as the surface density of (—NH₂) groups increases.

AN69 membranes are formed based on a copolymer prepared from sodium methallylsulfonate and acrylonitrile. It is possible to use other co-monomers instead of sodium methallylsulfonate. However, sodium methallylsulfonate is used as a specific, well known example for any such co-monomer throughout the present disclosure without the intention to limit the disclosure to said methallylsulfonate only. The molar ratio of acrylonitrile and sodium methallylsulfonate in the AN69 copolymer is in the range of from 90:10 and 99:1. According to one embodiment, the molar ratio is in the range of from 90:10 and 95:5. The AN69 membrane is hydrophilic because the numerous sulfonate groups attract water and create a hydrogel structure which provides high diffusive and hydraulic permeability. In the AN69 membrane the microstructure and the chemical composition offer a context for bulk adsorption of small proteins. The relatively high water content of the hydrogel generally makes the polymer chains easily accessible. The water content and the structure of acrylonitrile based hydrogel membranes of the prior art, specifically those based on sodium methallylsulfonate and acrylonitrile, are strongly influenced by the way the membranes are produced.

The AN69 membranes are generally produced by a phase inversion process making use of a hydrogel which is derived from a copolymer of acrylonitrile and sodium methallylsulfonate. The manufacturing process for AN69 hollow fiber membranes is based on high temperature spinning and the use of nitrogen as center medium when hollow fibers are produced. Hollow fibers are obtained by preparing a composition of a copolymer of acrylonitrile and sodium methallylsulfonate, N,N-dimethyl formamide (DMF), and glycerol and heating it to a temperature of from 110° C. to 150° C. before the composition enters the spinning nozzle, for example by means of a heating extrusion screw. According to one embodiment, the temperature is in the range of from 130° C. to 145° C. For the membranes in the Examples of the present disclosure, a temperature of 140° C. was chosen. The amount of the copolymer is generally adjusted to 34 to 36 wt.-%. For the membranes used in the Examples, the amount of copolymer was chosen to be 35 wt.-%. DMF or any other solvent which can be used, such as, for example, dimethylsulfoxide (DMSO) or N-methylpyrrolidone (NMP), is present in the composition in an amount of from about 50 to 58 wt.-%; and glycerol is present in an amount of from 6 to 16 weight-%. Of course, all components of the composition will add up to a total of 100%. According to one embodiment, the copolymer is present in the composition in an amount of 35 wt.-%, DMF is present in the composition in an amount of 52 wt.-%, and glycerol is present in an amount of 13 wt.-%. The composition is then passed through a spinneret. The extrusion is carried out in the presence of inert nitrogen as center medium. The fiber then enters a spinning bath.

The spinning bath is set up in a certain distance to where the fiber leaves the spinneret. The distance usually is in the range of from 0.8 to 1.9 m. The gap between nozzle and spinning bath contains ambient air at ambient temperature. Usually, the gap is located in a sealed cabin to prevent vapors from spreading. In the prior art, the spinning bath is adjusted to temperatures of from −4° C. to 20° C. Typical spinning bath temperatures for known AN69 membranes are in the range of from 6° C. to 20° C. For example, a standard spinning bath temperature for AN69 membranes is 10±2° C. The initial spinning bath usually consists of water. Optionally, additives such as H₂O₂ can be added in order to prevent bacterial growth. However, it is possible to add an organic solvent to the spinning bath. The solvents can be chosen from the same solvents which are used for forming the initial polymer composition.

Subsequent to the submersion into the spinning bath, the fiber may be subjected to an operation of stretching at a temperature of about 90° C. to 100° C., generally at about 95° C. The stretching operation is performed while the fiber is still immersed in water, and the desired temperature can be achieved by heating the water accordingly. The stretching can be achieved by adjusting the speed of the uptake rollers onto which the fibers are transferred from the spinning bath. It is known that the stretching ratio is impacting the formation and ratio of certain amorphous membrane zones and pseudo-crystalline zones of certain membrane types (Xi et al.: Polymers for advanced Technologies 19 (2008) 1616-1622). Stretching adds to the alignment of amorphous zones which in turn increases the structural integrity of the resulting membrane. An increased stretching ratio may further increase the Lp of the membrane to a certain extent.

The expression “Lp” or “hydraulic permeability” as used herein refers to the permeability of the membrane to water or an aqueous solution (saline solution), hereinafter referred to as “liquid”. Hydraulic permeability expresses how readily the liquid can move across a membrane and has units of volume of liquid per unit area of membrane per unit time per unit driving force.

The stretching ratio is defined by the take-up speed of the second roller which is higher compared to the take-up speed of the first roller. According to the present disclosure, the ratio preferably is in a range of from 3.6 to 4.5. According to a specific embodiment, the stretching ratio is in a range of from 3.6 to 4.1. Stretching ratios of 5 or higher are undesirable because they may result in damaged or torn fibers. High stretching ratios may also result in a phenomenon referred to as “crystallization under constraint”, which refers to an extended reorganization of the amorphous zone, leading to a behavior which is typical rather for impermeable crystalline zones.

It was found that a membrane having high bacteria adsorption can be obtained by treating a membrane based on a copolymer prepared from sodium methallylsulfonate and acrylonitrile with a suspension of a polycationic polymer in colloidal form. Membranes according to the present disclosure which are characterized by a polycationic polymer in colloidal form grafted to their surface are a further object of the present disclosure.

In the context of the present disclosure, the term “colloidal suspension” means a suspension comprising particles of the polycationic polymer dispersed in a continuous phase, preferably an aqueous phase. The size of said particles of the polycationic polymer (mean diameter) is 500 nm or less; for instance, in the range of from 10 to 500 nm; e.g., from 50 to 200 nm; or even from 80 to 120 nm; for example, some 100 nm.

According to the present disclosure, the membranes are treated by ionic grafting of a polycationic polymer selected from the group consisting of polyamines, such as cationic polyaminoacids and/or polyimines, comprising polylysine, polyarginine, polyethyleneimine (PEI) and copolymers and mixtures thereof. According to a specific embodiment of the present disclosure, said polycationic polymer is PEI.

The ionic grafting process uses a suspension of the poly-cationic polymer in an acidic medium. The suspension comprises at least one organic polyacid, allowing the poly-cationic polymer to take said colloidal form.

The organic polyacid may be a polycarboxylic acid, in particular a polycarboxylic acid comprising at least three carboxylic acid groups, preferably an organic tribasic acid. The polycarboxylic acid in one embodiment is citric acid.

According to one embodiment, the molecular mass of the polycationic polymer is greater than the cutoff threshold of the base membrane comprised of a copolymer prepared from sodium methallylsulfonate and acrylonitrile.

According to another embodiment of the present disclosure, the polycationic polymer comprises chains of sufficient size not to traverse the base membrane. Thus, the poly-cationic polymer, before being transformed into colloidal form, may comprise—chains with sufficient size (steric hindrance) not to traverse the base membrane.

This causes the polycationic polymer to be bound essentially to the surface of the membrane by ionic bonding. Consequently, the quantity of polycationic polymer necessary to treat the surface of the base membrane is moderate when a bulk treatment of the base membrane by penetration of the polycationic polymer into the base membrane is not sought.

In one embodiment, the polycationic polymer is prepared by ultrafiltration of a solution of the polycationic polymer through a semi-permeable membrane having a cutoff threshold which is greater than or equal to that of the base membrane. This allows chains of the polycationic polymer having a size smaller than the pores of the base membrane to be eliminated.

In one embodiment, the polycationic polymer is polyethyleneimine (PEI) having a mass average molecular weight, before ultrafiltration, in the range of from 500 to 1,000 kDa, for instance, approximately 750 kDa.

However, this ultrafiltration step may not be necessary as, when the polycationic polymer (of any molecular size) is transformed into colloidal form, the size of the polymer aggregates increases and the size obtained may then be sufficient not to penetrate the base membrane.

A colloidal suspension of the polycationic polymer can be obtained by mixing a first solution comprising the poly-cationic polymer with a second solution comprising at least one organic polyacid.

To obtain the colloidal suspension of the polycationic polymer, it is advantageous to carefully select the ratio of the concentration of the polycationic polymer and the organic polyacid. The ratio of the concentration of the poly-cationic polymer in the suspension to the concentration of organic polyacid in the suspension is such that it can produce a suspension in the colloidal form. As an example, in the implementation in which the polycationic polymer is polyethyleneimine and the polyacid is citric acid, said ratio of the concentration for PEI and citric acid is in the range 0.9 to 1.1.

In one embodiment, the base membrane is manufactured in a first step and then treated with a suspension of the poly-cationic polymer in colloidal form in a second step. Treatment with the suspension may be carried out on the surface, more particularly on the “apparent surface” (i.e. the apparent visible surface) but also optionally on the “developed surface” (i.e. on the apparent surface and the surface of the pores inside the base membrane).

In the case in which the apparent surface alone is treated, this is termed “surface treatment” of the base membrane. In the case in which the apparent and developed surfaces are treated, this is termed “bulk treatment” of the base membrane.

The membrane of the invention has a surface concentration for the polycationic polymer in the colloidal form in the range of from 1 to 20 mg/m², for instance, 1 to less than 15 mg/m², or 5 to less than 15 mg/m², or even 8 to 12 mg/m².

The membrane of the present disclosure may be a flat sheet membrane or a hollow fiber membrane. According to one aspect of the present disclosure, the membrane is a hollow fiber membrane which is composed of a homogeneous and symmetrical polyelectrolytic hydrogel derived from a copolymer of acrylonitrile and methallylsulfonate. Flat sheet membranes can also be prepared according to methods known in the art.

The hollow fibers according to the present disclosure have an internal diameter of from approximately 50 to approximately 260 μm. According to one embodiment of the disclosure, inner diameter will be in the range of from 180 to 250 μm. The wall thickness will generally be in the range of from 35 to 60 μm, preferably in a range of from 40 to 50 μm.

As mentioned above, it is a further object of the present disclosure to provide a hollow fiber membrane useful for producing a device for removing bacteria from liquids. According to one aspect of the present disclosure, the hollow fiber is composed of a homogeneous and symmetrical polyelectrolytic hydrogel as described above.

The surface area of a device comprising hollow fiber membranes according to the present disclosure may vary, but will usually be in a range of from 0.2 to 4 m², e.g., 1.0 to 2.3 m². Devices comprising the membrane of the present disclosure can be assembled as known in the art. Sterilization of the devices will normally be done using ETO.

A method for manufacturing a membrane for the removal of bacteria from a fluid comprises the following steps:

a) providing a base membrane essentially comprised of a first polymer carrying anionic or anionizable groups;
b) providing a suspension in an acid medium comprising a polycationic polymer carrying cationic or cationizable groups which are capable of forming an ionic bond with the anionic or anionizable groups of the first polymer, the polycationic polymer being in the colloidal form;
c) bringing the suspension into contact with at least a part of the surface of the base membrane.

In one embodiment of the membrane manufacturing method, the polycationic polymer comprises PEI and the organic polyacid comprises citric acid. The concentration of PEI in the suspension is in the range of from 0.01 to 0.2 g/l, e.g., 0.01 to 0.15 g/l; the ratio of PEI/citric acid concentrations by weight is 0.7 to 1.3, preferably 1.0.

Step c) is carried out by continuously circulating the suspension containing the polycationic polymer over at least one of the surfaces of the semi-permeable base membrane to be coated. In one embodiment, the suspension is produced by prior mixing of a solution of the polycationic polymer with a solution of an organic polyacid or by in-line mixing of the solutions just before treatment. In one embodiment, flow rate during treatment is 50 to 500 ml/min, for instance, 100 to 200 ml/min. The treatment can be performed in an open circuit or a closed circuit; liquid flow can be unidirectional or bidirectional. In one embodiment, duration of the treatment is from 1 to 30 min, e.g., from 5 to 10 min.

In one embodiment, glycerol is purged from the base membrane before the surface treatment in step c). For removal of glycerol, an aqueous liquid is circulated through the membrane.

The present disclosure also pertains to a method for manufacturing a device for the removal of bacteria from a liquid, e.g., a biological liquid, the device comprising a housing defining a first compartment and a second compartment, the first compartment being provided with an inlet and an outlet and being intended for the circulation of liquid to be treated, the second compartment being intended for the circulation of dialysate or filtrate and being provided with an outlet and optionally with an inlet, the two compartments being separated by a membrane, the method comprising the following steps:

a1) providing a membrane comprising a copolymer of acrylonitrile and methallylsulfonate;
a2) assembling the various components of the device, in particular mounting the membrane in the housing;
b1) providing a suspension in an acidic medium containing a polycationic polymer carrying cationic or cationizable groups which can form an ionic bond with the anionic or anionizable groups of the copolymer of acrylonitrile and methallylsulfonate, the second polymer being in the colloidal form;
b2) bringing the suspension into contact with at least a first part of the surface of the membrane intended to be brought into contact with the liquid to be treated;
b3) in case steps b1 and b2 are carried out after step a2, rinsing and/or purging the device of the solution containing the polycationic polymer; optionally followed by rinsing the membrane to eliminate excess non-bound polycationic polymer;
c1) optionally, sterilizing the device thus obtained.

In step c1), a sterilization mode is applied which does not negatively impact the chemical properties of the membrane surface, in order to maximize performance of the device. One suitable method is sterilization with ethylene oxide (ETO).

In one embodiment, the membrane, which may be a flat sheet membrane or a bundle of hollow fibers, is glycerolized at the end of step a1, hence the need to deglycerolize before step b2 defined above. Optionally, the membrane is again glycerolized after step b3.

The present disclosure also concerns the use of a membrane or a device of the present disclosure to remove bacteria from a liquid. In one embodiment, the liquid is a liquid intended to be injected into a patient. In one embodiment, the liquid is water. In another embodiment, the liquid is an aqueous solution or an aqueous suspension. Examples include substitution fluids, as well as solutions or suspensions comprising drugs, solutions or suspensions comprising electrolytes, solutions or suspensions comprising nutrients, etc. In still another embodiment, the liquid is plasma. In a further embodiment, the liquid is blood.

The present disclosure also concerns the use of a membrane or a device of the present disclosure in the treatment of septic syndrome by adsorption of bacteria present in a biological fluid that has been extracted from a patient suffering from septic syndrome; the biological fluid being blood or plasma.

The present disclosure also concerns the use of a membrane or a device of the present disclosure to remove bacteria from blood or plasma by extracorporeal circulation.

It is known that the AN69 type membranes have a remarkable ability to immobilize certain uremic toxins, including larger middle molecules, to their surface by adsorption. The membranes of the present disclosure can efficiently be used to remove living bacteria from a patient. Due to their specific characteristics, they additionally are capable of removing toxins of an extended range of molecular weight of the toxins, encompassing molecules which are generally referred to as middle molecules. The term “middle molecules”, as it is used in the context of the present disclosure, refers to molecules having a molecular weight between 15 kDa and 60 kDa, specifically to molecules having a molecular weight between 15 kDa and 45 kDa, even though in the prior art this expression is sometimes used for a broader range of molecules.

The membranes of the present disclosure and devices comprising said membranes, apart from being useful for removal of bacteria from blood or plasma in hemodialysis or hemodiafiltration treatment as mentioned before, may also be used for the treatment of chronic kidney disease patients who will benefit from the extended range of molecules which can be removed by the membrane. Due to the aforementioned adsorption capacities which allow the removal of an extended range of molecules, comprising molecules of up to about 60 kDa, combined with significantly improved convective properties, the membranes and devices of the present disclosure can be especially beneficially used in CRRT. Continuous renal replacement therapy (CRRT) is any extracorporeal blood purification therapy designed to substitute for impaired renal function over an extended period, and intended to be applied for up to 24 hours a day. CRRT is a modality specifically designed for treating ICU patients with acute kidney injury (AKI), especially in the case of hemodynamically unstable AKI patients. The membranes and devices of the disclosure can also be used in cascade filtration systems.

Devices according to the present disclosure may be used on known dialysis machines with blood flow rates of between 150 ml/min and 500 ml/min. Average blood flow rates will be in the range of between 200 and 500 ml/min. The devices comprising membranes according to the present disclosure can be used in hemodialysis as well as in hemodiafiltration (HDF) mode, including pre- and post-dilution.

The expression “HDF” as used herein refers to hemodiafiltration. While hemodialysis (HD) is primarily based on diffusion, thus relying on differences in concentration as the driving force for removing unwanted substances from blood, hemodiafiltration (HDF) also makes use of convective forces in addition to the diffusive driving force used in HD. Said convection is accomplished by creating a positive pressure gradient across the dialyzer membrane. Accordingly, blood is pumped through the blood compartment of the dialyzer at a high rate of ultrafiltration, so there is a high rate of movement of plasma water from blood to dialysate which must be replaced by substitution fluid that is infused directly into the blood line. Dialysis solution is also run through the dialysate compartment of the dialyzer. Hemodiafiltration is used because it may result in good removal of both large and small molecular weight solutes. The substitution fluid may be prepared on-line from dialysis solution wherein the dialysis solution is purified by passage through a set of membranes before infusing it directly into the blood line.

It will be readily apparent to one skilled in the art that various substitutions and modifications may be made to the disclosure disclosed herein without departing from the scope and spirit of the disclosure.

### EXAMPLES

### a) Fractionation of PEI

PEI was fractionated to eliminate the smallest polymer chains (with a low steric hindrance) which may penetrate into the pores of the semi-permeable membrane to be treated and traverse it. The procedure comprised the following steps:
1. A solution comprising 70 g/kg PEI with a mass average molecular mass of 750 kDa (LUPASOL P, BASF SE) in distilled water was prepared;
2. Said solution was circulated in a closed circuit, through the blood compartment of a capillary dialyzer provided with a membrane comprising PAES and PVP (Polyflux® 210H, GAMBRO Dialysatoren GmbH, 72379 Hechingen, Germany; effective surface 2.1 m²) at a flow rate of 700 ml/min;
3. Simultaneously with step 2, ultrafiltration at a flow rate of 180 ml/min was performed, and distilled water was added to the tank at the same flow rate.

### b) Preparation of AN69 Hollow Fibers

Hollow fibers as shown herein were obtained using a gelification process that consists of processing a collodion composed of acrylonitrile and sodium methallylsulfonate (91:9) (35 wt.-%), DMF (52 wt.-%) and glycerol (13 wt.-%), as further described above. The spinning nozzle temperature was adjusted to 140° C. The center medium was nitrogen (inert gas). The default spinning bath temperature was set to 10° C. The distance of the spinneret to the spinning bath was set to 1 m. Stretching was performed at about 95° C.

### c) Preparation of Hollow Fiber Modules

Modules comprising the hollow fiber membranes obtained in step b) were prepared. The modules had a length of 24 cm. The fibers were isolated from each other by using polyurethane glue at both ends. The fibers were re-opened at the potted ends by cutting off the tips of the bundle. The effective membrane surface area (A) of the fibers amounted to 1.5 m².

### d) Control (0 mg/kg PEI)

Modules as prepared in step c) were sterilized with ETO.

### e) Comparison Example 1 (HeprAN Type)

The modules were first rinsed with water for 3.5 min to remove residual glycerol. Subsequently, they were perfused with an aqueous solution comprising 200 mg/kg polyethyleneimine (PEI) in citric acid (ratio PEI/Ac=1) at pH 4 for 7 min at a flow rate of 200 ml/min. They were rinsed with water for 150 sec; and then they were perfused with an aqueous solution of 100 UI/ml heparin at a flow rate of 100 ml/min for 5 min 30 sec. Then they were rinsed again with an aqueous solution of glycerol (60% w/w) for 4 min at a flow rate of 250 ml/min. The modules finally were sterilized with ETO.

### f) Example 1 (10 mg/kg PEI)

Modules as prepared in step c) were first rinsed with water for 3.5 min to remove residual glycerol. Subsequently, they were perfused with an aqueous solution comprising 10 mg/kg polyethyleneimine (PEI) in citric acid (ratio PEI/Ac=1) at pH 4 for 7 min at a flow rate of 200 ml/min. Then they were rinsed again with an aqueous solution of glycerol (60% w/w) for 4 min at a flow rate of 250 ml/min. The modules finally were sterilized with ETO.

The concentration of PEI grafted on the membrane was determined to be 1 mg/m². To determine the concentration of PEI grafted on the membrane, a sample of fibers was taken from one of the modules prepared. The primary amine groups of the PEI grafted on the membrane were reacted with 2,4,6-trinitrobenzene sulfonic acid hydrate and the concentration of PEI was determined photometrically at 340 nm.

### g) Example 2 (50 mg/kg PEI)

Modules as prepared in step c) were first rinsed with water for 3.5 min to remove residual glycerol. Subsequently, they were perfused with an aqueous solution comprising 50 mg/kg polyethyleneimine (PEI) in citric acid (ratio PEI/Ac=1) at pH 4 for 7 min at a flow rate of 200 ml/min. Then they were rinsed again with an aqueous solution of glycerol (60% w/w) for 4 min at a flow rate of 250 ml/min. Finally, the modules were sterilized with ETO.

The concentration of PEI grafted on the membrane was determined to be 5 mg/m².

### h) Example 3 (200 mg/kg PEI)

Modules as prepared in step c) were first rinsed with water for 3.5 min to remove residual glycerol. Subsequently, they were perfused with an aqueous solution comprising 200 mg/kg polyethyleneimine (PEI) in citric acid (ratio PEI/Ac=1) at pH 4 for 7 min at a flow rate of 200 ml/min. Then they were rinsed again with an aqueous solution of glycerol (60% w/w) for 4 min at a flow rate of 250 ml/min. Finally, the modules were sterilized with ETO.

The concentration of PEI grafted on the membrane was determined to be 20 mg/m².

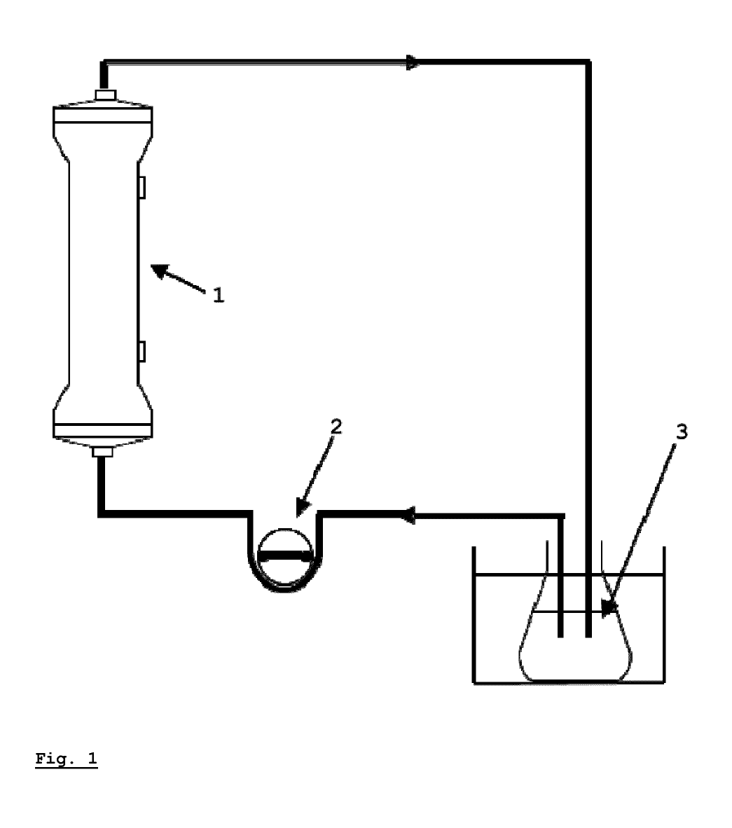

Bacteria retention of the modules obtained in steps d) to h) was tested using the setup shown in FIG. 1. The modules were first rinsed with 0.9% aqueous NaCl solution for 10 min to remove residual glycerol.

A volume of 500 ml of human plasma spiked with 5*10⁷ CFU Staphylococcus aureus was circulated from reservoir 3, maintained at room temperature, through a module 1 at a flow rate of Q_{Plasma}=200 ml/min using a peristaltic pump 2. After 2 hours of circulation, the experiment was stopped and the residual concentration of Staphylococcus aureus in the reservoir 3 was determined by blood culture using TSA growth media. From the residual concentration, the percentage of bacteria adsorbed on the membrane of the module was calculated.

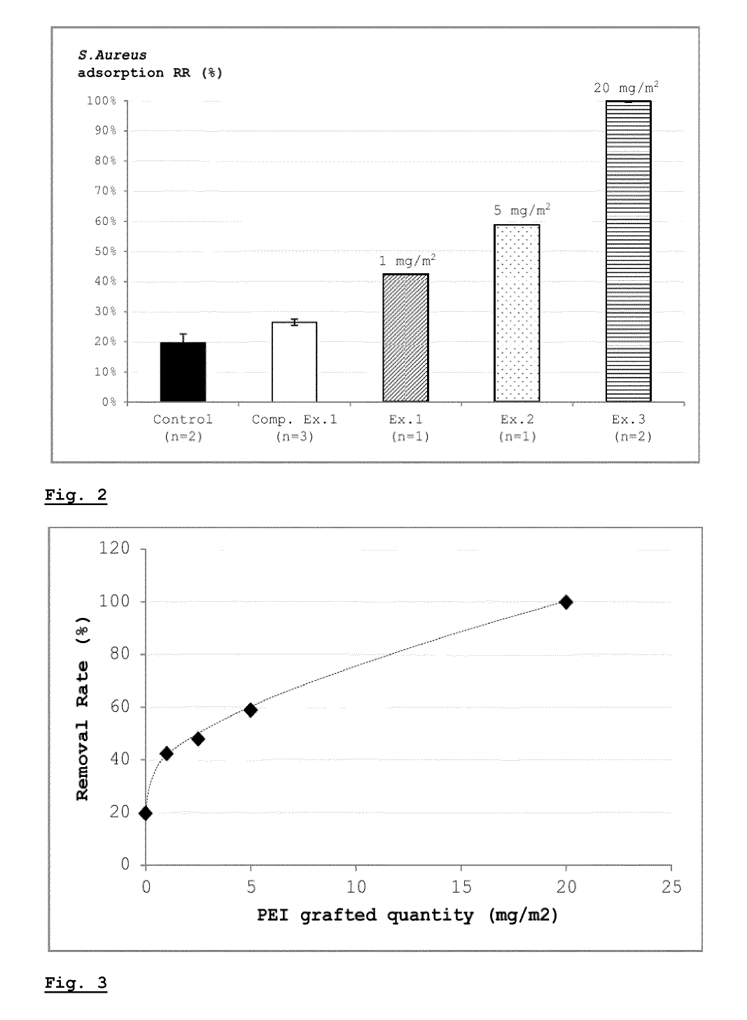

The results are depicted in FIG. 2. FIG. 3 shows a plot of the removal rate of Staphylococcus aureus (percent removal) as a function of the concentration of PEI grafted on the membrane (in mg/m²).

Using a model of gram-positive Staphylococcus aureus in human plasma in a closed-loop circuit, the experiments demonstrate the adsorptive capabilities of the grafted colloidal PEI.

### i) Example 4 (100 mg/kg PEI)

Modules as prepared in step c) were first rinsed with water for 3.5 min to remove residual glycerol. Subsequently, they were perfused with an aqueous solution comprising 100 mg/kg polyethyleneimine (PEI) in citric acid (ratio PEI/Ac=1) at pH 4 for 7 min at a flow rate of 200 ml/min. Then they were rinsed again with an aqueous solution of glycerol (60% w/w) for 4 min at a flow rate of 250 ml/min. Finally, the modules were sterilized with ETO.

The concentration of PEI grafted on the membrane was determined to be 10 mg/m².

In a further experiment, bacteria retention of both gram-positive Staphylococcus aureus and gram-negative Pseudomonas aeruginosa in human plasma was tested in a closed-loop circuit with the modules obtained in steps d) to i) using the setup shown in FIG. 1. As a comparison example, a filter module having an effective surface area of 1.5 m² and comprising an AN69 base membrane grafted with 30 mg/m² PEI and 4,500±1,500 UI/m² Heparin (oXiris®, Gambro) was also tested. The modules were first rinsed with 0.9% aqueous NaCl solution for 10 min to remove residual glycerol.

A volume of 500 ml of human plasma spiked with 5*10⁷ CFU Staphylococcus aureus and 5*10⁷ CFU Pseudomonas aeruginosa was circulated from reservoir 3, maintained at room temperature, through a module 1 at a flow rate of Q_{Plasma}=200 ml/min using a peristaltic pump 2. After 2 hours of circulation, the experiment was stopped and the residual concentration of Staphylococcus aureus and Pseudomonas aeruginosa, respectively, in the reservoir 3 was determined by blood culture using TSA growth media. From the residual concentration, the percentage of bacteria adsorbed on the membrane of the module was calculated. The results are summarized in Table 1.

TABLE 1
Bacteria retention rate of different filter modules

| | Retention rate [%] | | | | | |
|---|---|---|---|---|---|---|
| | Control | Ex. 1 | Ex. 2 | Ex. 4 | Ex. 3 | |
| | 0 | 1 | 5 | 10 | 20 | |
| | mg/m² | mg/m² | mg/m² | mg/m² | mg/m² | oXiris |
| S. aureus | 20 | 42 | 59 | 100 | 100 | 36 |
| P. aeruginosa | −7 | 0 | 15 | 84 | 92 | −9 |

1. A device for the removal of bacteria from a liquid, comprising a membrane based on a copolymer prepared from sodium methallylsulfonate and acrylonitrile, having a poly-cationic polymer in colloidal form with a mean particle diameter in the range of from about 10 to about 500 nm grafted to its surface, wherein the surface concentration of the poly-cationic polymer in the colloidal form is in the range of from about 5 mg/m² to less than about 15 mg/m²; with the proviso that the device does not comprise heparin.

2. The device of claim 1, wherein the membrane is a flat sheet membrane or a plurality of flat sheet membranes.

3. The device of claim 1, wherein the membrane is a plurality of hollow fiber membranes.

4. The device of claim 1, wherein the membrane has a surface area in the range of from about 0.4 m² to about 4.0 m².

5. The device of claim 1, wherein the polycationic polymer is a polyethyleneimine (PEI).

6. A process for manufacturing a device for the removal of bacteria from a liquid, the device comprising a housing defining a first compartment and a second compartment, the two compartments being separated by a membrane, with the proviso that the device does not comprise heparin, the method process comprising the following steps:
a) providing a membrane comprising a copolymer of acrylonitrile and methallylsulfonate;
b) mounting the membrane in the housing;
c) providing a suspension in an aqueous acidic medium containing citric acid and about 0.01 g/l to about 0.2 g/l of a polyethyleneimine (PEI) in colloidal form with a mean particle diameter in the range of from about 10 to about 500 nm, the weight ratio of PEI to citric acid being in the range of from about 0.7 to about 1.3;
d) bringing the suspension into contact with at least a first part of the surface of the membrane.

7. The process of claim 6, wherein step d) is performed by circulating the suspension over at least one of the surfaces of the membrane at a flow rate of from about 50 ml/min to about 500 ml/min for a time period of about 1 minute to about 30 minutes.

8. The process of claim 6, wherein the membrane is a flat sheet membrane or a plurality of flat sheet membranes.

9. The process of claim 6, wherein the membrane is a plurality of hollow fiber membranes.

10. The process of claim 6, wherein the membrane has a surface area in the range of from about 0.4 m² to about 4.0 m².

11. A process for the removal of bacteria from a liquid, the process comprising the step of circulating the liquid through a device for the removal of bacteria from a liquid, comprising a membrane based on a copolymer prepared from sodium methallylsulfonate and acrylonitrile, having a poly-cationic polymer in colloidal form with a mean particle diameter in the range of from about 10 to about 500 nm grafted to its surface, wherein the surface concentration of the poly-cationic polymer in the colloidal form is in the range of from about 5 mg/m² to less than about 15 mg/m²; with the proviso that the device does not comprise heparin.

12. The process of claim 11, wherein the liquid is selected from the group consisting of water, an aqueous solution, and an aqueous suspension.

13. The process of claim 12, wherein the liquid is a substitution fluid used in hemodiafiltration or hemofiltration.

14. The process of claim 12, wherein the liquid is an aqueous solution or an aqueous suspension.

15. The process of claim 11, wherein the liquid is blood or blood plasma.

16. The process of claim 14, wherein the aqueous solution or the aqueous suspension comprises one or more components selected from the group consisting of drugs, electrolytes, and nutrients.

17. The process of claim 11, wherein the membrane is a flat sheet membrane or a plurality of flat sheet membranes.

18. The process of claim 11, wherein the membrane is a plurality of hollow fiber membranes.

19. The process of claim 11, wherein the membrane has a surface area in the range of from about 0.4 m² to about 4.0 m².

20. The process of claim 11, wherein the polycationic polymer is a polyethyleneimine (PEI).
